(19) United States
(12) Patent Application Publication
(10) Pub. No.: US 2022/0096058 A1
(43) Pub. Date: Mar. 31, 2022

(54) System and Method for Time Series Collection of Excreted Sweat

(71) Applicant: Government of the United States, as represented by the Secretary of the Air Force, Wright-Patterson AFB, OH (US)

(72) Inventors: Sean Harshman, Fairborn, OH (US); Kraig Strayer, Fairborn, OH (US); Rhonda Pitsch, Springfield, OH (US)

(21) Appl. No.: 17/329,239

(22) Filed: May 25, 2021

Related U.S. Application Data
(60) Provisional application No. 63/083,387, filed on Sep. 25, 2020.

(51) Int. Cl. A61B 10/00; A61B 5/145; A61B 5/00

(52) U.S. Cl. CPC A61B 10/0064; A61B 5/14517; A61B 2560/04; A61B 5/6824; A61B 5/6831

(57) ABSTRACT

A system and method for collecting sweat for analytical evaluation over a period of time is disclosed. The system is configured so that discrete samples of sweat obtained during certain time intervals during the period of time can be segregated for analysis. The system includes a sweat collecting device that has a main body and a length of sweat collecting tubing that extends outward from a bore in the main body. A receptacle for collecting sweat, which may be in the form of a microfuge tube having a lid, is removably joined to the free end of the sweat collector tubing. The method includes attaching the sweat collection device over an area of a person's skin and collecting sweat from the person's skin using the sweat collection device. The sweat travels into the sweat collector tubing and then into the receptacle. During the period of time, when the desired sample has been collected, the receptacle can be removed and replaced with additional receptacles for collecting additional samples.

Pursuant to 37 C.F.R. § 1.78(a)(4), this application claims the benefit of and priority to prior filed co-pending Provisional Application Ser. No. 63/083,387, filed Sep. 25, 2020, which is expressly incorporated herein by reference.

### RIGHTS OF THE GOVERNMENT

The invention described herein may be manufactured and used by or for the Government of the United States for all governmental purposes without the payment of any royalty.

### FIELD OF THE INVENTION

The present invention relates generally to the collection of human sweat for analytical evaluation and, more particularly, to a system and method for collecting sweat over a period of time that enables time series samples to be collected.

### BACKGROUND OF THE INVENTION

Sweat has become an emerging biosource for biomarker discovery to sustain or enhance human performance. Typically, single large bulk sweat samples are collected for later off-line analysis. While useful, this sampling strategy limits the time-resolution of often-lengthy collection events and the utility of identified markers.

Historically, the most widely used method for sweat collections involves a single sample, obtained via a “patch” or other device, collected over the course of an entire experiment, such as an exercise or sweat stimulation regime. Therefore, the sample collected is only representative of the mean analyte concentration over the experimental time. As a result, changes of analytes, e.g. ions and metabolites, over the time course of the experiment are lost.

Therefore, a need exists for a system and method of collecting sweat over a period of time to better elucidate any changes in properties of the sweat and/or analyte concentration that may occur during the course of an experiment.

### SUMMARY OF THE INVENTION

While the invention will be described in connection with certain embodiments, it will be understood that the invention is not limited to these embodiments. To the contrary, this invention includes all alternatives, modifications, and equivalents as may be included within the spirit and scope of the present invention.

The present invention relates generally to the collection of human sweat for analytical evaluation and, more particularly, to a system and method for collecting sweat over a period of time that enables time series samples to be collected. The system is configured so that discrete samples of sweat obtained during certain time intervals during the collection period can be segregated for analysis.

The sweat collection system comprises a sweat collecting device and a plurality of receptacles for collecting sweat samples. The sweat collecting device comprises a main body having two surfaces comprising a sweat-collecting surface and an opposing outwardly-oriented surface. The sweat-collecting surface has a concave configuration which defines a rim lying wholly within a common plane. The main body has an axial bore extending between the sweat-collecting surface and the outwardly-oriented surface. A length of sweat collector tubing has one end joined to the main body at the axial bore and a free end that extends outwardly from the outwardly-oriented surface of the main body. The sweat collecting device further comprises a closure for joining to a receptacle for collecting sweat. The closure may have a hole therethrough, or on the side thereof, wherein the hole is sized to fit at least partially around the sweat collector tubing that is located adjacent to the free end of the sweat collector tubing. The sweat collection system and device comprise at least one receptacle for collecting sweat. The receptacle has a closed end and an open end. The receptacle is positioned so when the closure is placed over the open end of the receptacle, the free end of the sweat collector tubing is directed into the open end of the receptacle.

A method of collecting sweat from a person's skin for testing is also provided. The method comprises:
a) attaching a sweat collection device over an area of the person's skin, wherein the sweat collection device comprises:
   a main body having two surfaces comprising a sweat-collecting surface and an opposing outwardly-oriented surface, wherein the sweat-collecting surface has a concave configuration which defines a rim lying wholly within a common plane, and the main body has an axial bore extending between the sweat-collecting surface and the outwardly-oriented surface;
   a length of sweat collector tubing having one end joined to the main body at the axial bore and a free end extending outwardly from the outwardly-oriented surface of said main body;
   a closure for a receptacle, wherein the closure has a hole therethrough, wherein the hole is sized to fit at least partially around the sweat collector tubing adjacent the free end of the sweat collector tubing;
b) providing a receptacle for collecting sweat, wherein the receptacle has a closed end, an open end, and positioning the receptacle so that when the closure is placed over the open end of the receptacle, the closure is removably joined to the open end of the receptacle and the free end of the sweat collector tubing is directed into the open end of the receptacle; and
c) collecting sweat from the person's skin using the sweat collection device wherein sweat travels into the sweat collector tubing and then into the receptacle.

At various points during the collection period, when the desired sample has been collected, the receptacle can be removed and replaced with an additional receptacle for collecting an additional sample.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a perspective view of a prior art sweat collection device attached to a portion of a person's forearm.

FIG. 2 is a perspective view of the sweat collection device described herein attached to a portion of a person's forearm.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

The present invention relates generally to the collection of human sweat for analytical evaluation and, more particularly, to a system and method for collecting sweat over a period of time that enables time series samples to be collected.

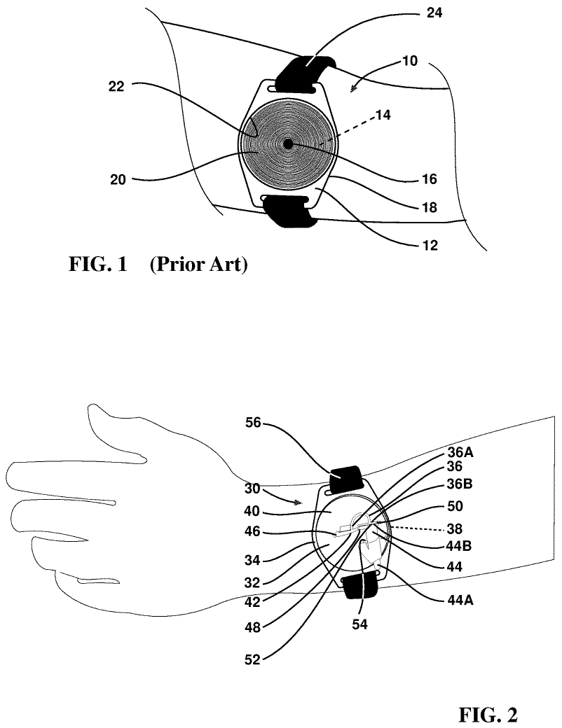

FIG. 1 shows a prior art sweat collection device 10 attached to a portion of a person's forearm. The prior art sweat collection device 10 shown is a MEGADUCT® sweat testing device sold by ELITechGroup of Logan, Utah, U.S.A. ELITechGroup also sells a similar, but smaller MACRODUCT® sweat collection device. These products and the procedure for using the same is described in greater detail in U.S. Pat. No. 4,383,529, Webster and U.S. Pat. No. 4,542,751, Webster, et al. The sweat collection device 10 is typically used in a process to determine the concentration of sodium and chloride in the sweat of children suffering from cystic fibrosis. U.S. Pat. No. 4,383,529, Webster discloses an iontophoretic electrode device that is used with an iontophoretic drug such as pilocarpine to induce sweating at a selected area of a person's skin prior to placing the sweat collection device against the person's skin. Certain improvements to these products are described in U.S. Pat. No. 6,198,953 B1, Webster, et al. and U.S. Pat. No. 9,226,730 B2, Briscoe, et al.

The MEGADUCT® sweat collecting device 10, as described in the aforementioned patents, comprises a solid body 12 having a concave means defined at a face thereof by a sweat-collecting surface 14 which gradually recedes from a rim lying wholly within a common plane. In FIG. 1, the sweat-collecting surface 14 is facing the wearer's skin. An axial bore 16 extends from the sweat-collecting surface to the opposite face 18 of the solid body. The maximum depth of the concave means is such that the skin and flesh of the flat area of a human body to which the device is applied will bulge into contact with the sweat-collecting surface 14 over substantially the entire area thereof when the rim is placed firmly against the skin of the flat area of the human body, leaving substantially no dead space in the interface between the bulged skin and the sweat-collecting surface 14. The body secretion pressure of sweat will force sweat through the interface to the axial bore 16. The axial bore 16 is adapted to connect with a length of flexible, sweat collector tubing 20 at the opposite face 18 of the solid body 12. The sweat collector tubing 20 is coiled in a flat, spiral formation and is positioned within a recessed receiving chamber 22 on the face 18 of the body 12 opposite the sweat-collecting surface 14. The solid body 12 is fastened against the flat area of a portion 26 of a human body such as by straps 24 so as to maintain firm placement of the rim and sweat-collecting surface 14 against the skin.

U.S. Pat. No. 4,542,751, Webster, et al. describes how the sample of sweat is obtained when using the MEGADUCT® sweat collection device after it is collected in the spiral tubing. The tubing 20 has to be disconnected from the axial bore 16. The sweat then has to be expelled from the tubing 20 into a testing vessel by inserting an elastomer bulb into one end of the tubing 20 and squeezing the bulb. As a result, all of the sweat collected in the tubing is mixed together in the testing vessel. Because of this, it is not possible to analyze samples of sweat obtained at different times during the collection period.

FIG. 2 shows the sweat collection system 30 of the present invention. The present invention constitutes an improvement over the MACRODUCT® and MEGADUCT® sweat collecting devices and current procedures for using the same in order to allow discrete samples of sweat obtained during certain time intervals to be collected.

The sweat collection system 30 of the present invention comprises a sweat collecting device 32 comprising a main body 34 and a length of sweat collector tubing (or “tube”) 36. The main body 34 has two surfaces comprising a sweat-collecting surface 38 and an opposing outwardly-oriented surface 40. The sweat-collecting surface 38 has a concave configuration which defines a rim lying wholly within a common plane. The main body 34 has an axial bore 42 extending between the sweat-collecting surface 38 and the outwardly-oriented surface 40.

The sweat collector tubing 36 may have any suitable length including, but not limited to about 40 mm. The sweat collector tubing 36 does not have to be long enough to store sweat therein. The sweat collector tubing 36 may have any suitable inside diameter that permits the transportation of sweat through the tubing. The sweat collector tubing 36 may, for example, have an inside diameter of about 0.86 millimeters. The sweat collector tubing 36 has a first end 36A that is joined to the main body 34 of the sweat collecting device 32 at the axial bore 42, and a free second end 36B extending outwardly from the outwardly-oriented surface 40 of the main body 34.

The sweat collection device 32 further comprises at least one receptacle 44 for collecting sweat. The receptacle 44 can be of any suitable configuration that allows sweat to be collected from the tubing 36 without loss (through evaporation, or otherwise) or contamination of the contents. The free end 36B of the sweat collector tubing 36 is positioned so that the sweat will be directed into the receptacle 44.

FIG. 2 shows that the receptacle 44 is in the form of a tube that has a closed end 44A, an open end 44B, and a lid or cap 46 for closing the open end 44B. The opening at the open end 44B of the receptacle 44 may be larger than the outside diameter of the sweat collector tubing 36. In some cases, the open end 44B of the receptacle 44 may be 2, 3, 4, 5, or more times larger than the outside diameter of the sweat collector tubing 36. The receptacle 44 may have relatively more rigid or inflexible sides than the sweat collector tubing 36. The receptacle 44 may, for example, be a test tube or a microfuge tube. The receptacle 44 shown in FIG. 2 is a microfuge tube having a width that tapers to a conical portion with a rounded closed end 44A. The lid or cap 46 can be separate from the microfuge tube, or it can be joined to the microfuge tube by a plastic hinge 48 adjacent to the open end 44B of the receptacle 44. The lid or cap 46 can have a snap fit interlocking closure to close the open end 44B of the receptacle 44.

The sweat collecting device 32 may further comprise a closure (or cover) 50 for the receptacle 44. The closure 50 provides space for the sweat to be transferred into the receptacle 44 through the tubing 36, but otherwise closes the open end of the receptacle 44 when sweat is being collected to prevent evaporation or loss. Any suitable type of closure 50 can be provided including, but not limited to stoppers, lids and caps. The closure 50 can comprise any suitable type of mechanism for joining to the receptacles 44 including, but not limited to: serving to plug the open end of the receptacle; forming a threaded connection with the receptacle; and providing a snap fit interlocking closure with the receptacle.

The closure 50 shown in FIG. 2 comprise a second lid or cap for a microfuge tube that has been modified by having a hole 52 therein (e.g., drilled therethrough). The hole 52 can be in any suitable portion of the closure 50 such as the middle of the closure, or it can be in the form of a recessed area at the edge of the closure 50, so long as the sweat collector tubing 36 can either pass through the closure 50 or adjacent to the closure and the closure 50 is able to close off the remainder of the opening 44B of the receptacle. The hole 52 may be sized to fit at least partially around the sweat collector tubing 36, preferably snuggly around the sweat collector tubing 36. The free second end 36B of the sweat collector tubing 36 may be inserted into or through the hole 52, and the tubing 36 may be joined to the closure 50 such as by gluing or other method. (If the hole 52 is large enough that it leaves a gap around the sides of the sweat collector tubing, and the gap is closed with glue or other material, the 50 may have an additional vent hole therein to assist in initiating the flow of sweat.

Joining the closure 50 to the sweat collector tubing 36 allows a series of receptacles 44 (such as microfuge tubes) to be easily swapped out during the sweat collection process. The closure 50 can be removed from one receptacle after sweat is collected therein, and then joined to an empty receptacle 44 by the snap fit interlocking mechanism. The receptacles 44 may be thought of as being removably joined to the free end 36B of the sweat collector tubing 36 (indirectly through the closure 50) since the sweat collector tubing 36 will be positioned to dispense into the open end of the receptacles 44.

A plurality of such sweat collection receptacles 44 can be provided for the sweat collection system for sequentially connecting to the closure 50. A given receptacle 44 may be considered to be part of the sweat collecting device 32 when the receptacle 44 is in use. When not in use, a given receptacle 44 may be considered to be a separate element that is part of the sweat collection system 30. These receptacles 44 may be referred to as first, second, and third, etc. receptacles. The receptacles 44 typically do not have air evacuated from the inside thereof so that the air inside the tubes is at atmospheric pressure.

FIG. 2 shows that one of the receptacles 44 may be removably joined to the main body 34 of the sweat collecting device 32. The receptacle 44 can be removably joined to any suitable portion of the sweat collecting device 32 in any suitable manner. In FIG. 2, a holder 54 for a receptacle 44 is joined to the outwardly-oriented surface 40 of the main body 34 of the sweat collecting device 32. Any suitable type of holder 54 may be used. The holder 54 may comprise a structure in the nature of a sleeve with an opening therein into which the receptacle 44 can be inserted. In FIG. 2, the holder 54 comprises at least a portion of a larger component such as a larger size microfuge tube that the receptacle 44 may be inserted into and easily removed therefrom. The larger microfuge tube may be modified in any suitable manner to serve as the holder 54 (such as by cutting off the tip at the closed end). In other cases, the receptacle 44 may be removably joined to the main body 34 of the sweat collection device 32 by a holder 54 comprising a piece of tape.

The sweat collection system of the present invention can be made by modifying a MEGADUCT® sweat collecting device by trimming the sweat collector tubing that is provided. The main body 34 of the sweat collection device 32 may, thus, have any of the properties of the solid body of the MEGADUCT® device described above. The remaining tubing, approximately 40 mm in length, is then inserted into a hole drilled into a cap of a microfuge tube. The sweat collector tubing is joined to the cap such as by gluing. The cap which serves as closure 50 is then attached to the open end 44B of microfuge tube 44. The tubes can be exchanged as the experiment allows. In other embodiments, it is possible to drill holes into the cap or lids for each microfuge tube that will be used as a receptacle. However, this would be less desirable because it would require more labor for drilling the holes, and would render such additional lids non-functional for storing the sweat after it has been collected.

The method of using the sweat collection system 30 is as follows. The sweat collection device 32 is attached over an area of a person's skin. The collection device 32 can be attached in any suitable manner such as with tape, an extensible bandage, or with straps 56. The sweat collection device 32 is joined to a subject's body and sweat is stimulated as the experimental design allows. The participants may be asked to generate a sweat sample for collection either through stimulation, heat acclimation, or exercise. Stimulation with an iontophoretic electrode device may be used, but is not necessary if sweat will be generated without that procedure.

A first sweat sample is collected from the person's skin over a first time interval using the sweat collection device 32 wherein sweat travels into the sweat collector tubing 36 and then into the first receptacle 44. The first receptacle 44 can be removed from the closure 50 that is connected to the tubing 36, and a cap without a hole therein can be placed over the open end 44B of the receptacle 44. The closure 50 can then be placed on a second clean microfuge tube and collection of a second sample over a second time period proceeds. The procedure can be repeated with a third, fourth, fifth, etc. receptacle as needed to collect samples throughout a sweat protocol. Exchange of the microfuge tubes, during the experiment, allows for time series collection. The system is, thus, configured so that discrete samples of sweat obtained during certain time intervals during the overall period of time can be segregated for analysis.

Sample analysis may be performed immediately after collection, or at a later time. The samples can be analyzed for any suitable properties or analytes. It may be desirable to determine the quantity of sweat that is generated in response to: certain activities; the person's heart rate during an exercise regime; ambient temperature, etc. at various times throughout the sample collection period. The samples can be analyzed for any substances including, but not limited to the presence of biomarkers as described in U.S. patent application Ser. No. 16/431,326, filed on Jun. 4, 2019 and published as U.S. Patent Application Publication US 2019/0365298 A1, Harshman, et al.

Various alternative embodiments of the system and method for collecting sweat over a period of time are possible. It is understood that several of the aforementioned steps of the method can be performed in any suitable order. For instance, the method may involve attaching the sweat collection device 32 over a person's skin before or after sweating is induced. In addition, the receptacle 44 can be removably joined to the closure 50 before or after the sweat collection device 32 is attached over a person's skin. In another, but less desirable embodiment, a microwell plate could be used instead of the plurality of microfuge tubes. In such an embodiment, the free end of the sweat collector tubing would be moved from one well to another throughout the collection. In addition, although collection and analysis of sweat from humans is primarily of interest, the system and method of the present invention could be used with any other biological subject that is capable of producing and excreting sweat.

The system and method for collecting sweat over a period of time described herein can provide a number of advantages. It should be understood, however, that these advantages need not be required unless they are set forth in the appended claims. The present invention provides for sampling of sweat, in time, in sufficient quantities for off-line biochemical analysis and ultimately biomarker discovery. This allows the sweat to be tested for changes in analytes over the entire course of the sample collection.

The terms “join” and “joined”, as used herein, encompass configurations in which an element is directly secured to another element by affixing the element directly to the other element; configurations in which the element is indirectly secured to the other element by affixing the element to intermediate member(s) which in turn are affixed to the other element; and configurations in which one element is integral with another element, i.e., one element is essentially part of the other element. The term “joined” includes both those configurations in which an element is temporarily joined to another element, or in which an element is permanently joined to another element.

The disclosure of all patents, patent applications (and any patents which issue thereon, as well as any corresponding published foreign patent applications), and publications mentioned throughout this description are hereby incorporated by reference herein. It is expressly not admitted, however, that any of the documents incorporated by reference herein teach or disclose the present invention.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

What is claimed is:

1. A sweat collection system comprising:
a sweat collecting device comprising:
a main body having two surfaces comprising a sweat-collecting surface and an opposing outwardly-oriented surface, wherein said sweat-collecting surface has a concave configuration which defines a rim lying wholly within a common plane, wherein said main body has an axial bore between the sweat-collecting surface and the outwardly-oriented surface;
a length of sweat collector tubing having one end joined to said main body at said axial bore and a free end extending outwardly from the outwardly-oriented surface of said main body; and
a closure for a receptacle, wherein the closure has a hole therethrough, wherein the hole is sized to fit at least partially around the sweat collector tubing adjacent the free end of the sweat collector tubing; and
a receptacle for collecting sweat, said receptacle having a closed end and an open end, wherein the receptacle is positioned so when the closure is placed over the open end of the receptacle and is removably joined to the receptacle, the free end of the sweat collector tubing is directed into the open end of the receptacle.

2. The sweat collection system of claim 1 wherein the receptacle is a microfuge tube.

3. The sweat collection system of claim 2 wherein the closure is a cap for a microfuge tube.

4. The sweat collection system of claim 1 wherein the receptacle is removably joined to the main body of the sweat collecting device.

5. The sweat collection system of claim 4 further comprising a holder joined to the outwardly-oriented surface of the main body of the sweat collecting device, wherein said holder comprises a sleeve with an opening for receiving said receptacle, and said receptacle is removably joined to the main body of the sweat collecting device when it is inserted into said holder.

6. The sweat collection system of claim 1 comprising additional receptacles for sequentially connecting to the free end of the sweat collector tubing by joining the receptacles to said closure.

7. A method of collecting sweat from a person's skin for testing, said method comprising:
a) attaching a sweat collection device over an area of the person's skin, wherein said sweat collection device comprises:
a main body having two surfaces comprising a sweat-collecting surface and an opposing outwardly-oriented surface, wherein said sweat-collecting surface has a concave configuration which defines a rim lying wholly within a common plane, wherein said main body has an axial bore extending between the sweat-collecting surface and the outwardly-oriented surface;
a length of sweat collector tubing having one end joined to said main body at said axial bore and a free end extending outwardly from the outwardly-oriented surface of said main body; and
a closure for a receptacle, wherein the closure has a hole therethrough, wherein the hole is sized to fit at least partially around the sweat collector tubing adjacent the free end of the sweat collector tubing;
b) providing a receptacle for collecting sweat, said receptacle having a closed end, an open end, and positioning said receptacle so that when the closure is placed over the open end of the receptacle, the closure is removably joined to the open end of the receptacle and the free end of the sweat collector tubing is directed into the open end of the receptacle; and
c) collecting sweat from the person's skin using said sweat collection device wherein sweat travels into said sweat collector tubing and then into said receptacle.

8. The method of claim 7 wherein the receptacle is a microfuge tube.

9. The method of claim 7 further comprising removably joining the receptacle to the outwardly-oriented surface of the main body of the sweat collecting device.

10. The method of claim 7 wherein said receptacle is a first receptacle, said method further comprising:
providing at least one additional receptacle for sequentially connecting to the closure, wherein each additional receptacle has a closed end and an open end;
removing said first receptacle from said closure; and
joining the open end of an additional receptacle to said closure.

11. The method of claim 10 wherein:
said first receptacle comprises a first microfuge tube which has a lid joined to a portion of said receptacle adjacent the open end for closing said open end, wherein said lid is in an open position when said first microfuge tube is joined to said closure;
said at least one additional receptacle comprises a second microfuge tube which has a lid joined to a portion of said receptacle adjacent the open end for closing said open end, wherein said lid is in an open position; and
said method further comprises removing said first microfuge tube from said closure, closing the open end of said first microfuge tube with said lid, and joining the open end of said second microfuge tube to said closure.
